(19) United States
(12) Patent Application Publication
(10) Pub. No.: US 2011/0230766 A1
(43) Pub. Date: Sep. 22, 2011

(54) ULTRASOUND IMAGING MODALITY IMPROVEMENT

(75) Inventor: Andrew Medlin, Thebarton (AU)

(73) Assignee: SIGNOSTICS LIMITED, Torrensville, South Australia (AU)

(21) Appl. No.: 13/062,949

(22) PCT Filed: Oct. 2, 2009
(86) PCT No.: PCT/AU2009/001307

§ 371 Date: Mar. 9, 2011

(30) Foreign Application Priority Data
Oct. 9, 2008 (AU) 2008905241

(51) Int. Cl. A61B 8/14

(52) U.S. Cl. 600/459

(57) ABSTRACT

An ultrasound imaging device is disclosed including a transducer with two elements having different operating frequencies, which are arranged to be coaxial. The device has four modes of use, the first mode of use being use of one element for imaging at a first depth range, the second mode of use being use of the other element for imaging at a different depth range, the third mode of use being tissue harmonic imaging, and a fourth mode of use being demodulation imaging. A single image produced by the imaging device includes data contributed by two or more of the modes of use.

### FIELD OF THE INVENTION

The present invention relates to a method and apparatus for the transmission and reception of acoustic waves for the purpose of imaging.

### BACKGROUND OF THE INVENTION

A variety of equipment and methods exist for imaging using ultrasound energy, with applications in medical imaging, industrial non-destructive testing, and underwater imaging. In general, these systems transmit an ultrasound pulse and wait for returned echoes generated by changes of impedance of the structures being imaged. The returned echoes are processed and displayed on a screen as an image, a graph, or some other format. The quality of the image and data generated is dependent on many factors, but two important factors are the beam width at the point of reflection and the length of the pulse transmitted. Narrow beams provide improved lateral resolution and short pulses provide improved axial resolution.

Axial resolution (also known as the depth, linear, longitudinal and range resolution) is the minimum distance in the beam direction between two reflectors which can be identified as separate echoes. The axial resolution is slightly more than half the spatial pulse length, which is the number of waves in the transmitted ultrasound pulse multiplied by their wavelength.

Transducer bandwidth and pulse length are related. Theoretically, only infinite sine waves have a single frequency. The beginning and end of an ultrasound pulse introduce a range of frequencies; the shorter the pulse, the wider its frequency spectrum. A low bandwidth transducer will respond to a short voltage pulse with a relatively long lasting vibration, emitting ultrasound with a narrow bandwidth, but a long pulse length. This gives poor axial resolution.

Ultrasonic transducers have a resonant frequency at which the amplitude of vibration is maximal. About this resonant frequency is a band of nearby frequencies which are also passed. The transducer is most effective at generating an ultrasonic signal at the resonant frequency, and also most sensitive to receiving signals at this frequency.

The broader the bandwidth, the more effective the transducer at producing short discrete pulses giving better image resolution, but this also makes it less sensitive overall to receive signals. On the other hand, narrower bandwidth transducers produce longer signals giving lower resolution, but are more sensitive to receiving echo signals.

A broadband transducer will emit a short pulse of ultrasound consisting of a broad range of frequencies, which will improve axial resolution, but there are limitations to the width of passband which can be achieved with practical transducers. There is also the problem that increasing transducer bandwidth leads to reduced efficiency in driving the transducer.

Demodulation imaging as described in U.S. patent application Ser. No. 12/307,305, which is hereby incorporated in its entirety by reference, may also be used to improve axial resolution.

The choice between resolution and sensitivity is determined by the practical application of the device for which the transducer is to be designed. For 2D pulse-echo imaging the optimum lies with the shortest possible pulse which still maintains enough sensitivity to capture images from the desired depth. For continuous wave (CW) Doppler, maximal sensitivity is required and therefore a narrow band transducer would be used.

In conventional ultrasound the receive frequency is the same as the transmit frequency because at this frequency the echo signal amplitude is largest. Typically the same transducer crystal is used for both transmit and receive in pulse-echo imaging applications. CW Doppler implementations may use separate transmit and receive crystals of the same resonant frequency.

In pulse-echo imaging the choice of operating frequency is determined by a trade off between signal penetration depth and resolution. Higher frequencies produce a shorter and therefore higher resolution pulse length, but attenuate at a higher rate as they travel through the target material. A lower operating frequency generates a signal that is lower resolution but also penetrates to greater depths.

Clinically, different frequency transducers are used for distinct clinical applications. High frequency is typically used for shallow depth applications, while low frequency is used for scanning to greater depth.

There exists a need to obtain improvements in ultrasound imaging technology to enable improved image quality without increased cost.

### DISCLOSURE OF THE INVENTION

A dual-frequency ultrasonic transducer is proposed in which one or more high frequency elements and one or more low frequency elements are used together in order to implement clinical ultrasound scanning. The multiple elements are provided in a simple construction which allows for a low cost device which is able to implement relatively complex imaging modalities.

In one form of this invention there is proposed an ultrasound transducer including at least one first element having a first resonant frequency and at least one second element having a second resonant frequency, wherein the first frequency is higher than the second frequency and the first and second elements are arranged to be co-axial.

The provision of a multiple frequency capability allows for an ultrasound imaging device including the transducer to implement ultrasound imaging modalities beyond simple B mode imaging.

There is provided a dual frequency system having the capability to both transmit and receive at each of two distinct frequencies, and able to be configured for operation in any combination of those capabilities.

Using a low frequency transducer element to transmit an acoustic search signal and high frequency transducer elements to receive the reflected acoustic signal gives the capability for nonlinear harmonic imaging.

Low frequency transmission combined with low frequency reception allows deep conventional imaging.

Nonlinear demodulation imaging is achieved by using the high frequency transducer elements to transmit the acoustic search signal and the low frequency transducer to receive the reflected echoes.

Using the high frequency transducer elements to both transmit and receive allows for shallow conventional imaging with relatively high resolution.

In a further form of the invention there is provided an ultrasound imaging device including the proposed transducer which implements tissue harmonic imaging.

Preferably, the ultrasound imaging device implements demodulation imaging.

The simplicity of the transducer allows the ultrasound scanning device to be small and to consume little power. This allows the device to be a personal ultrasound, that is, to be of a size to be carried by a user in a pocket or around the neck.

Preferably, the ultrasound imaging device weighs less than one kilogram.

### BRIEF DESCRIPTION OF THE DRAWINGS

FIG. 1 shows a schematic representation of a hand held ultrasound apparatus incorporating an embodiment of the invention.

FIG. 2 shows the operation of a piston transducer of the prior art.

FIG. 3 shows the manner of use of non-axial transducers of the prior art.

FIG. 4 shows a diagrammatic illustration of a transducer of the invention.

FIG. 5 is a frequency domain plot of signals in a non-linear medium.

FIG. 6 shows time and frequency domain plots for a short signal pulse.

FIG. 7 shows time and frequency domain plots for a longer signal pulse.

FIG. 8 is a plot illustrating energy conversion efficiency against frequency.

FIG. 9 is a diagram illustrating the use of curved transducer faces to manipulate focal points.

### BEST MODE FOR CARRYING OUT THE INVENTION

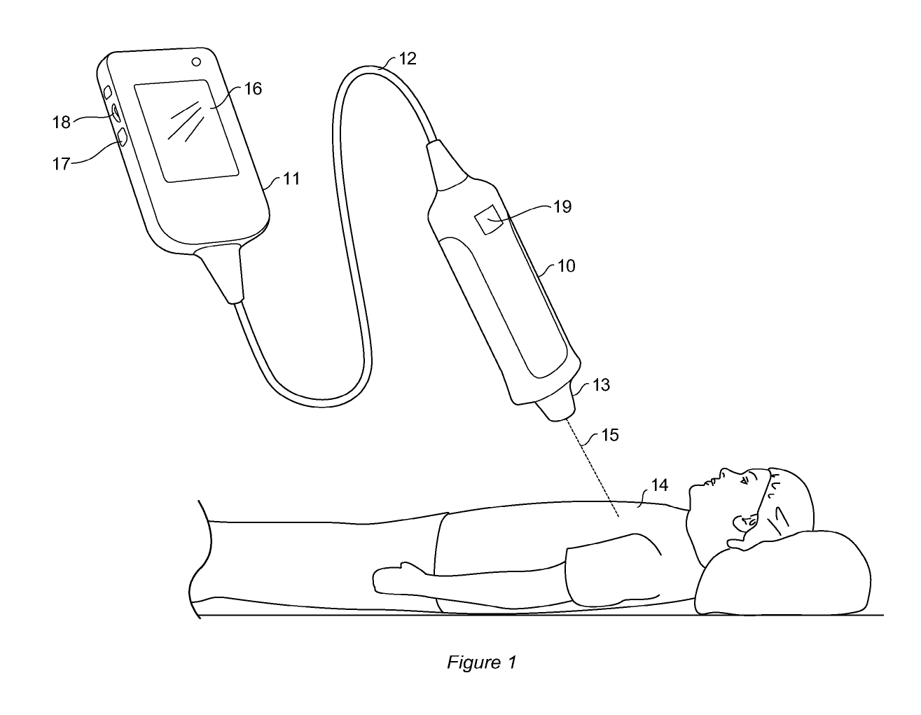

FIG. 1 shows a handheld ultrasound transmission, reception and analysis device, schematically represented in use in a medical diagnostic setting. The illustration is not to scale.

There is a hand held ultrasonic probe unit 10, a display and processing unit (DPU) 11 with a display screen 16 and a cable 12 connecting the probe unit to the DPU 11. The DPU includes a thumbwheel 18, which is able to be rotated up and down and to be pressed inward to the body of the DPU. These movements provide control signals for the user interface. There is also provided a further interface control button, the back button 17.

The probe unit 10 includes an ultrasonic transducer 13 adapted to transmit pulsed ultrasonic signals into a target body 14 and to receive returned echoes from the target body 14.

In this embodiment, the transducer is adapted to transmit and receive in only a single direction at a fixed orientation to the probe unit, producing data for a single scanline 15. The transducer has a plurality of elements which operate at a plurality of frequencies.

The probe unit further includes an orientation sensor 19 capable of sensing orientation or relative orientation about one or more axes of the probe unit. Thus, in general, the sensor is able to sense rotation about any or all of the axes of the probe unit.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

Since the distance between the mounting point of the sensor 19 and the tip of the transducer 13 is known, it would also be possible to implement the sensor with one, two or three accelerometers.

The DPU includes a touchscreen user interface device 16. This gives the user control of a user interface which allows parameters for an ultrasound scan to be set. Further user input devices may be provided. These include but are not limited to, a scroll wheel 18, numeric or alpha numeric keypad and voice recognition means.

The user interface may be used to set any parameters for the scan to be undertaken, and to view and enhance the resultant scan image. It may be used to determine the mode of use of the transducer for the scan being made.

In use the user rotates the probe as required to sweep the ultrasound beam over the desired area, keeping linear displacement to a minimum. The reflected echoes of the ultrasound beam are received by the transducer.

In embodiments where rotation about all axes is not sensed, the user will also keep rotation about unsensed axes, that is axes about which rotation is not detected by the sensor of the embodiment, to a minimum.

At the same time, data is received from the orientation sensor 19. This is the rotation about the sensed axes of the probe unit. It may be the angular change in the position of the probe unit since the immediately previous transducer pulse, or the orientation of the probe unit in some defined frame of reference. One such frame of reference may be defined by nominating one transducer pulse, normally the first of a scan sequence, as the zero of orientation.

The sensor data and the output signal of the transducer are combined to give a scanline. A scanline is a dataset which comprises a sequential series of intensity values of the response signal combined with orientation information. A scan dataset is a plurality of sequentially received scanlines.

The invention may be embodied in a hand held medical diagnostic device as shown in FIG. 1, or in any other configuration in which ultrasound equipment is made or used.

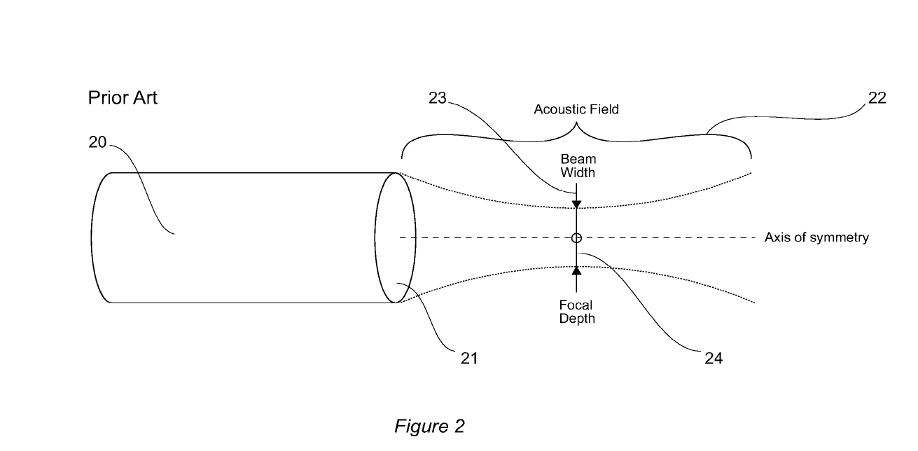

FIG. 2 shows a piston transducer of the prior art, adapted for imaging along a given line with a circular, piston like transducer. The transducer has a cylindrical body 20, with a circular face 21. This circular face may optionally have a surface curvature to realise a particular focal depth. This style of transducer generates an acoustic field 22 with axial symmetry.

The axisymmetric field is characterised by a beam width 23 and a focal depth 24, which depend on the parameters of operating frequency, piston diameter, and radius of curvature of the piston face. These parameters may be adjusted to alter the acoustic field pattern. The relationship between the transducer geometry and the acoustic field also holds for the receiving transducer. Therefore for good reception of the receive signal it is beneficial for the receiver to be co-located and aligned along the transmitter axis. This is obviously the case when the transmitter and receiver are one and the same crystal.

The configuration in which the transmitter and receiver are one and the same crystal is beneficial for signal reception, but is limited in that it can only operate at one frequency. Changing to a different operating frequency would involve physically replacing the whole transducer.

Some imaging modalities exploit tissue properties by receiving at a different frequency to the transmitter. Typically this receive frequency is far enough removed that it cannot be detected by the transmitter and necessitates a separate receiver optimised to the desired reception frequency.

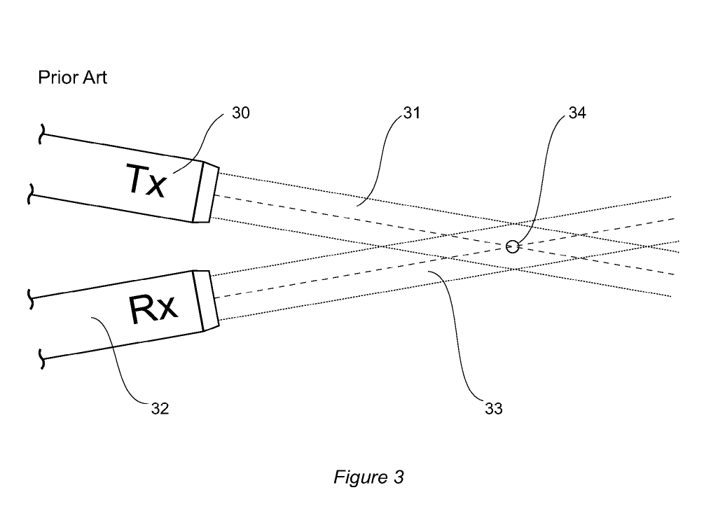

When distinct transmit and receive transducers or transducer elements are required, their geometrical arrangement becomes a design consideration. As shown in FIG. 3, a transmit transducer element 30 with an acoustic field 31 may be physically located adjacent to a receive transducer element 32 with an acoustic field 33. A common focus 34 can be achieved but the acoustic fields are not axis aligned.

It is beneficial for the transmit and receive transducers to share a common axis. This avoids complications that arises if the receive transducer does not share an axisymmetric view of the acoustic field with the transmitter. Avoidance of such complications leads to simplicity and therefore reduced cost.

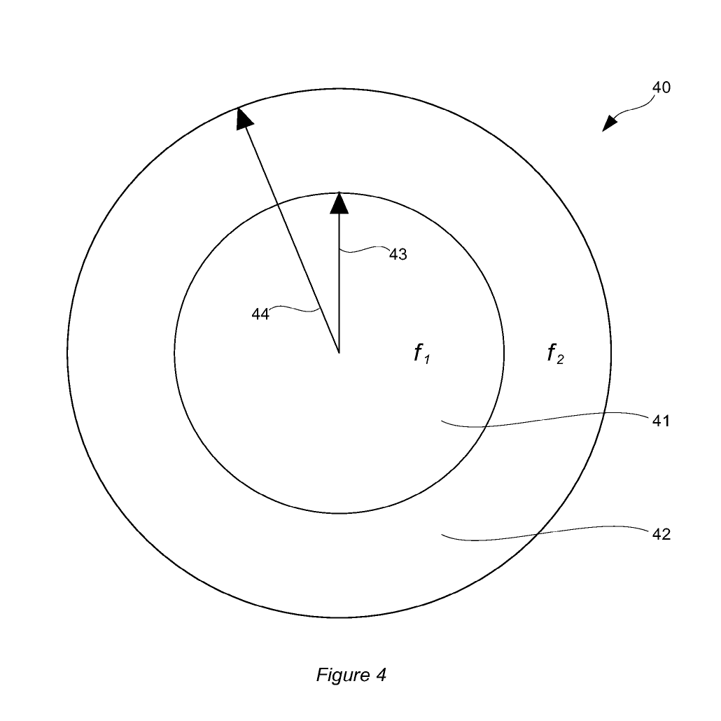

FIG. 4 shows the simplest axisymmetric arrangement for a multi element transducer being a piston transducer 40 having two elements, an inner circular element 41 and an outer annular element 42. The inner element has a frequency of operation f₁ and the outer element has a frequency of operation of f₂.

This is the construction of transducer 13. The dual-frequency ultrasonic device of FIG. 1 uses the two transducer elements tandem to achieve various imaging modes. In other embodiments, the transducer may have more than one element operating at each of the frequencies.

Electronics and control logic for controlling the two elements to transmit and receive the two operating frequencies may be located in the probe unit 10 or the DPU 11, or may be split between the two units.

The design of the two elements of the transducer is of significance. In the linear approximation to signal propagation through tissue, each frequency component in the signal is independent of every other. In the absence of absorption, the field pattern is dictated purely by diffraction effects and the frequency power spectrum remains constant. When absorption is present, the amount of power in the signal at each component frequency decreases with propagation distance. The rate of attenuation varies according to a power law relationship, resulting in a preferentially higher attenuation rate a high frequencies compared to low frequencies:

α=α₀f_{c}ⁿ

Here, α is the attenuation rate at a component frequency f. α₀ is a material dependent reference attenuation and n is the exponent. The component frequency f is a component of the frequency spectrum of the signal transmitted by an ultrasound transducer for imaging purposes.

Following this power law relationship, the peak in the signal shifts down to lower frequencies with propagation depth. In the theoretical situation where the attenuation response of the material into which the signal is propagating is linear, the power at each frequency is always decreasing.

In practice there is often significant non-linearity in the mechanisms that modulate the frequency spectrum of the signal. Human tissue has a non-linear response. Two such nonlinear effects are harmonic resonance and self-demodulation.

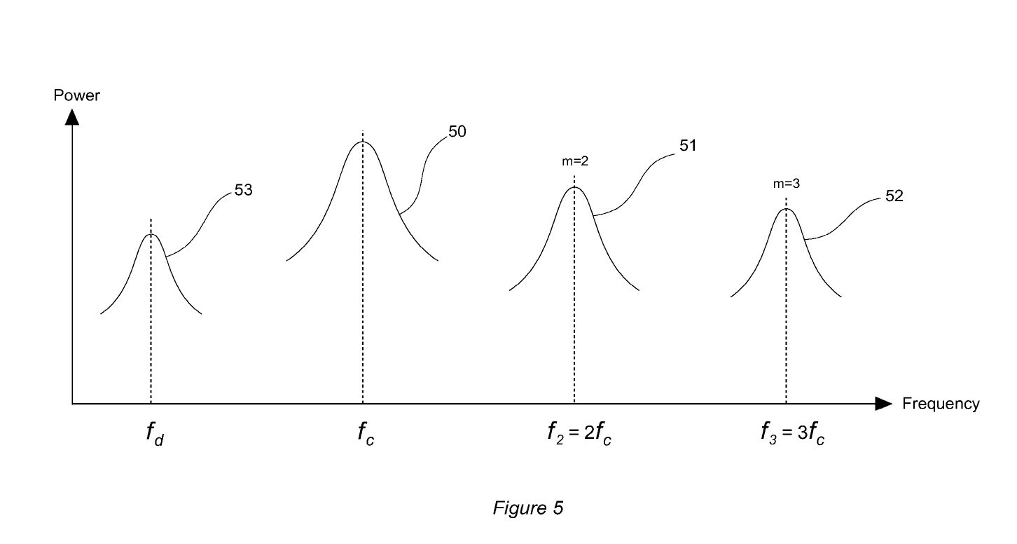

FIG. 5 shows a frequency domain plot of a generic transmitted signal 50, having a frequency f_{c}.

Harmonic resonance produces increasing power at higher frequencies than the operating frequency, typically at whole multiples of the operating frequency. This is shown in FIG. 5 where there is a local peak 51 in the signal power at a frequency f₂ which is 2f_{c}. A further peak 52 appears at frequency f₃ which is 3f_{c}. The effect appears when the transmitted signal amplitude is sufficiently large in nonlinear media.

Self-demodulation produces increasing power at lower frequencies than the operating frequency. This lower frequency may be referred to as a subharmonic frequency. FIG. 5 shows a power peak 53 at the demodulation frequency f_{d}.

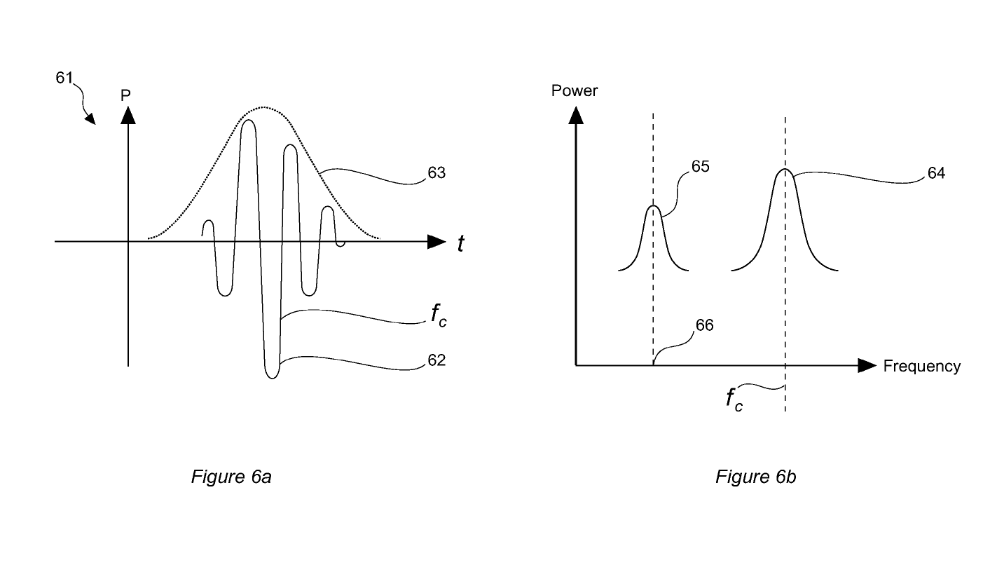

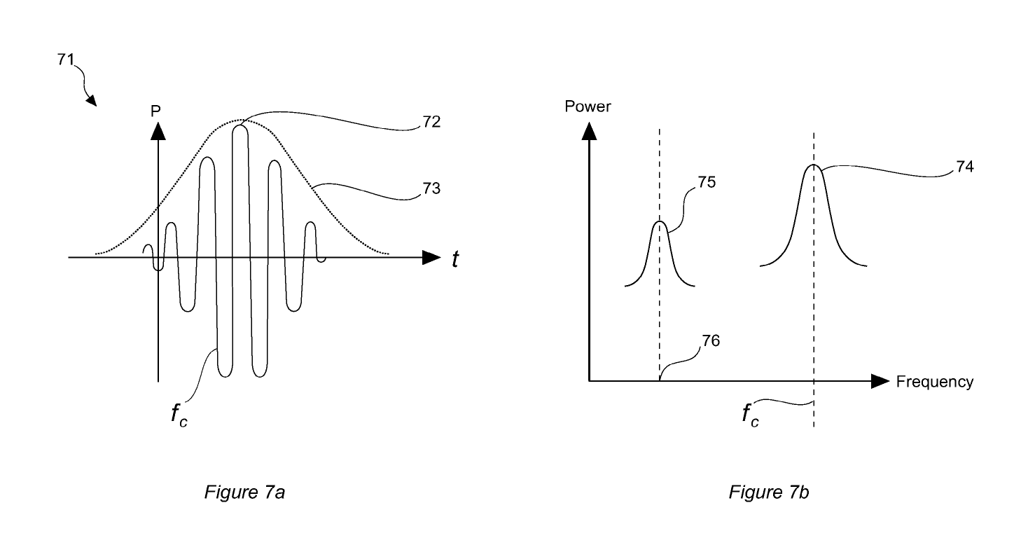

The frequency f_{d} about which the demodulation signal is centred is related to the envelope of the original transmit signal. A measure of control over the demodulation frequency can be obtained by adjusting the envelope of the transmit pulse, as illustrated in FIG. 6 and FIG. 7.

FIG. 6a shows a short transmit pulse 61 having a centre frequency f_{c} 62. The signal is of approximately three cycles, which is close to the lowest practical limit for producing a signal for ultrasound imaging. The envelope 63 of the signal 61 is narrow.

The corresponding demodulated signal is shown in FIG. 6b. There is a strong peak 64 at the transmit frequency. There is also a peak 65 at a demodulation frequency f_{d} 66. The demodulation frequency is approximately ⅓f_{c}

FIG. 7a shows a longer transmit pulse 71 having a centre frequency f_{c} 72. The signal is of approximately six cycles. The envelope 73 of the signal 71 is broad.

The corresponding demodulated signal is shown in FIG. 7b. There is a strong peak 74 at the transmit frequency. There is also a peak 75 at a demodulation frequency f_{d} 76. The demodulation frequency is approximately ⅙f_{c}.

The demodulation frequency is related to the reciprocal of the number of cycles of the transmit pulse. Longer pulses lead to lower demodulation frequencies.

Nonlinear imaging modalities exploit this nonlinear behaviour of the target medium. Human tissue has a non-linear response, making these modalities useful for medical imaging.

Both the harmonic and self-demodulated signals are generated within the medium itself, in proportion to the source signal amplitude and the material-dependent degree of nonlinearity. Consequently the spatial intensity of a nonlinear frequency component can be used as a sensitive indicator of material properties for improved material characterisation. For example, used in concert with specially designed contrast agents nonlinear imaging can provide high contrast images of perfusive structures in human or animal tissue.

Since the nonlinear signal band has little or no overlap with the transmit signal, and is generated within the body of the target medium, nonlinear imaging modalities offer advantages such as reduced speckle and better signal-to-noise ratio.

Harmonic imaging filters the field at a receive frequency f₂>f_{c}, which being higher than the transmit frequency offers improved lateral resolution.

Demodulation imaging filters the field at a receive frequency f_{d}<f_{c}, which being lower than the transmit frequency offers imaging to greater depth because the signal absorption is lower. Particular choice of the envelope of the transmit signal also allows exceptional axial resolution to be achieved with demodulation imaging by generation of a waveform having the shortest possible pulse length, a single cycle.

Referring to the transducer of FIG. 4, in general, one of the frequencies f₁, f₂ is higher than the other. Since the resonant frequencies of the transducers are fixed, there is a fixed ratio

$$m = \frac{{f}_{high}}{{f}_{low}}$$

between the high frequency f_{high} and the low frequency f_{low}. The ratio m is chosen to give the best imaging performance. FIG. 5 shows that power peaks occur at harmonic intervals. The transducer is designed to take advantage of these power peaks.

Harmonics occur at integer multiples of the carrier frequency, so m must be equal or nearly equal to an integer >=2. Exact equality is not required because the transducers have a finite bandwidth (and therefore sensitivity not only at the resonant frequency, but also near the resonant frequency) and because the peak frequency drifts downwards due to the effect of attenuation. The higher the harmonic, the weaker it is, so the greatest power for harmonic imaging is available when m=2.

In demodulation imaging, as shown in FIG. 6 and FIG. 7 the ratio m between f_{high} and f_{low} relates to the brevity of the transmit pulse. A ratio of m=2 implies just 2 cycles of the carrier frequency within the envelope of the pulse, which is just beyond the limit of what is practically achievable. A ratio of 2.5-3.0 is more realistic.

Therefore it is understood that the frequency ratio m is a fixed frequency multiple common to harmonic imaging and demodulation imaging, and that it is selected judiciously so that the demodulation frequency f_{d} is at or near f_{c}/m=f_{high}/m, and the harmonic frequency fₕ is at or near f_{c}*m=f_{low}*m.

In the preferred embodiment, m is chosen to be 3, but it can be seen that other values are possible.

In practical conventional imaging, it is common and beneficial to have a low frequency and a high frequency transmitter in order to facilitate both deep and shallow imaging. Values of the ratio m from 2 to 5 would be typical in order to cover a range of scan depths and image resolutions.

In general it is desirable to use as high a frequency as possible in all imaging modes in order to maximise the image resolution. However, higher resolution comes at the price of greater signal loss through attenuation, so that in practice a balance is sought between resolution and maximum imaging depth.

The maximum required scan depth, the properties of the target material and the signal sensitivity of the embodying hardware would determine the actual choice of the low frequency transducer. For example, for tissue an application might require imaging to 30 cm depth and a particular embodiment might then require a low frequency transducer no greater than 2 MHz.

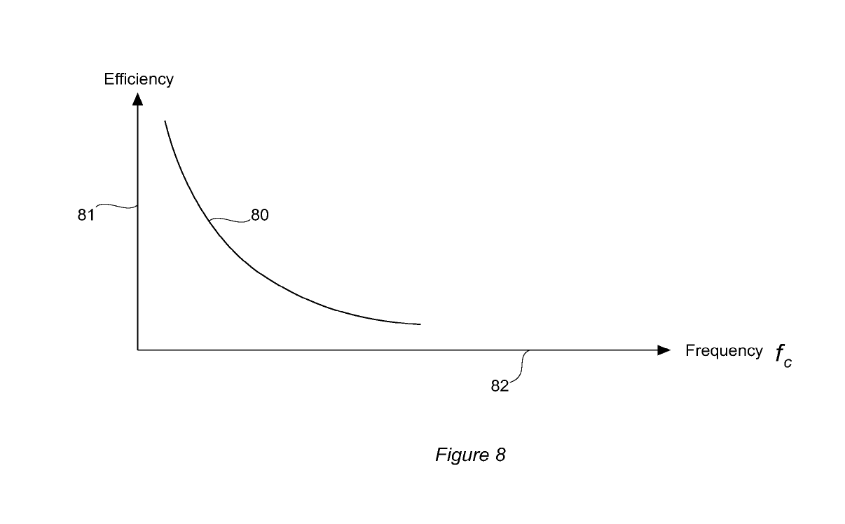

From the perspective of the current invention, the depth range capability above needs to be considered simultaneously with the ability to generate nonlinear harmonic and demodulated signals of sufficient amplitude. FIG. 8 illustrates the trendline 80 of nonlinear conversion efficiency 81 as a function of transmit frequency 82.

The plot of FIG. 8 illustrates that nonlinear signals of greater amplitude are generated at lower frequencies, because more of the carrier signal is converted to nonlinear signal. This trend is counter to the preference to increase the frequency in order to maximise the resolution.

Therefore choice of transducer frequencies is a balance between the need for a lower frequency to maximise the nonlinear signal with the need for a higher frequency to maximise resolution.

In a preferred embodiment, f_{low}=3 Mhz. Using m=3, this gives f_{high}=9 MHz. The preferred embodiment provides useful performance in medical imaging. There is a useful range of parameter values in which performance needs can be reconciled.

Two principles are used to assist in designing the dimensions of the elements and designating the frequencies.

First, the signal collection area of the receive element needs to be comparable to the transmit element in order to collect sufficient signal. Since both elements are capable of transmit and receive in different operating modes, this tells us that the high and low frequency elements must be similar in total area.

Second, the focal regions of the high and low frequency elements must overlap as much as possible.

Equating the areas of both transducer elements shown in FIG. 4 yields the inner radius in terms of the total radius R:

$${r}_{1} = \frac{R}{\sqrt{2}}$$

This shows that to have sufficient collection area in the simple configuration the inner radius must r₁ be 1/\sqrt{2} times, or about 70% of, the outer radius R.

Considering equivalent foci, the natural focal length F of a flat faced piston transducer is

$$F = \frac{{fr}^{2}}{c}$$

where c is the acoustic propagation speed, f is the frequency and r is the piston radius.

Ignoring the factor c which has no bearing on equivalence, considering an inner element of radius r₁ and an outer element of radius R, we get

$$\begin{matrix}{F}_{2} = {f}_{2} {R}^{2} \\ = 2 {f}_{2} {r}_{1}^{2} \\ = 2 ( \frac{{f}_{2}}{{f}_{1}} ) {f}_{1} {r}_{1}^{2} \\ = 2 ( \frac{{f}_{2}}{{f}_{1}} ) {F}_{1}\end{matrix}$$

where we have applied the area equivalence formula to get F₂ in terms of F₁.

If F₂ is to be in the same order as F₁ then f₂<f₁. Thus, in the preferred embodiment the high frequency element is the inner element 41, and the outer element 42 is the low frequency element.

A further advantage of having the outer, annular element as the low frequency element is that the near-field region of an annular element transmitted signal field is weak and is therefore unsuitable for the shallowest imaging. Since high frequency is best suited to shallow imaging, it is advantageous to use the inner circular element for the high frequency transmit signal to ensure a uniform signal in the shallow near-field region.

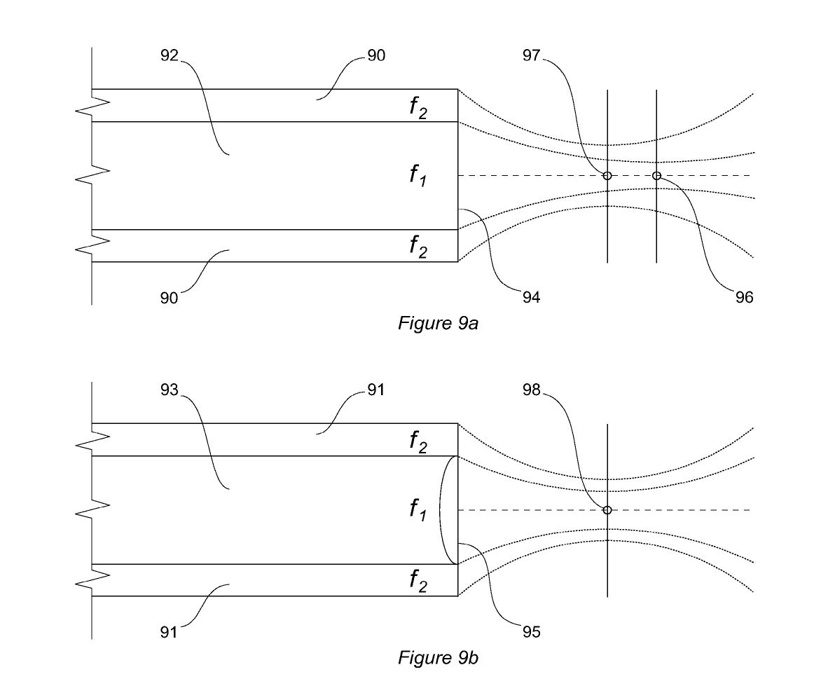

For improved image quality it is desirable to reduce discrepancies in the positions of the foci of the two elements as much as possible. In an embodiment this is achieved by addition of geometrical curvature of the face of the transducer element, as illustrated in FIG. 9.

FIG. 9a shows a two element piston transducer with a uniformly flat face. There is an annular outer transducer element 90, which has a focal point 97 at an operating frequency f₂. This surrounds a cylindrical transducer element 92 which has a flat face 94. This cylindrical element has a focal point 96 at an operating frequency f₁. It can be seen that the focal points of the two transducer elements are not co-incident.

In FIG. 9b, there is again an annular transducer element 91, with a focal point 98 at an operating frequency f₂. This again surrounds a cylindrical transducer element 93. In this case the face 95 of the cylindrical element is concave, with the concavity being selected such that the focal point 98 at an operating frequency at an operating frequency f₁ is coincident with the focal point of the annular transducer.

The transducer thus designed may produce scanline data from one element for imaging at one depth range and from the other element for imaging at another depth. Using a low frequency transducer element to transmit an acoustic search signal and high frequency transducer elements to receive the reflected acoustic signal provides capability for nonlinear harmonic imaging. Low frequency transmission combined with low frequency reception allows deep conventional imaging. Nonlinear demodulation imaging is achieved by using the high frequency transducer elements to transmit the acoustic search signal and the low frequency transducer to receive the reflected echoes. Using the high frequency transducer elements to both transmit and receive allows for shallow conventional imaging with relatively high resolution.

The transducer is able to perform imaging which would otherwise require the expense and inconvenience of multiple transducers.

In an embodiment, scanline data or processed image data from any of the modes of use described may be combined to give a composite image with greater depth, clearer focus or improved axial resolution over a greater depth range than could be achieved for an image made using any one mode alone.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-20. (canceled)

21. A transducer for an ultrasound imaging device having at least one first element having a first resonant frequency and at least one second element having a second resonant frequency, the first frequency being higher than the second frequency, wherein the transducer is controlled to have a first mode of use being use of the first element for imaging at a first depth range and to have a second mode of use being use of the second element for imaging at a second depth range, the first depth range being on average shallower than the second depth range and to have a third mode of use being tissue harmonic imaging and to have a fourth mode of use being demodulation imaging.

22. The transducer of claim 21 wherein a single image produced by the imaging device includes data contributed by two or more of the modes of use.

23. The transducer of claim 21 wherein the first and second elements are arranged to be approximately co-axial.

24. The transducer of claim 21 wherein the total areas of the first and second elements are approximately equal.

25. The transducer of claim 23 wherein the first element is substantially disk shaped and the second element is shaped substantially as an annulus surrounding said first element.

26. The transducer of claim 23 wherein the second element is substantially disk shaped and the first element is shaped substantially as an annulus surrounding said second element.

27. The transducer of claim 25 wherein the face of the first element is shaped such that the focal point of ultrasound energy transmitted by the second element is located at approximately the same point in space as the focal point of ultrasound energy transmitted by the first element.

28. The transducer of claim 21 wherein the ratio of the first frequency to the second frequency is in the range 2 to 5.

29. The transducer of claim 21 wherein the ratio of the first frequency to the second frequency is approximately 3.

30. The transducer of claim 21 wherein the first frequency is in the range 8 to 10 MHz and the second frequency is in the range 2 to 4 MHz.

31. The transducer of claim 21 wherein the first frequency is 9 MHz and the second frequency is 3 MHz.

32. The transducer of claim 21 wherein the first element comprises a plurality of sub-elements.

33. The transducer of claim 21 wherein the second element comprises a plurality of sub-elements.

34. An ultrasonic imaging device including the transducer of claim 21 including control circuitry adapted to control the elements of the transducer and to process a receive signal from the transducer, to produce scanlines incorporating echo data from the first element, scanlines incorporating echo data from the second element, scanlines incorporating tissue harmonic imaging and scanlines incorporating demodulation imaging.

35. The ultrasonic imaging device of claim 34 wherein a single image produced by the imaging device includes scanline data contributed by two or more of the modes of use.

36. The ultrasonic imaging device of claim 34 further including an inertial sensor.

37. An ultrasonic imaging device including the transducer of claim 21 wherein the ultrasound imaging device weighs less than one kilogram.

38. An ultrasonic imaging device including the transducer of claim 21 wherein the ultrasound imaging device weighs less than five hundred grams.
